(19) United States
(12) Patent Application Publication
(10) Pub. No.: US 2022/0047423 A1
(43) Pub. Date: Feb. 17, 2022

(54) WAVEFRONT HIGHER ORDER CORRECTION OF IOLS USING REFRACTIVE INDEX MODIFICATION

(71) Applicant: AMO Development, LLC, SANTA ANA, CA (US)

(72) Inventors: Richard Hofer, Santa Cruz, CA (US); Alexander Vankov, Mountain View, CA (US); Jenny Wang, Mountain View, CA (US); David A. Dewey, Sunnyvale, CA (US); Georg Schuele, Portola Valley, CA (US)

(21) Appl. No.: 17/057,683

(22) PCT Filed: Apr. 9, 2020
(86) PCT No.: PCT/IB2020/053423

§ 371 Date: Nov. 22, 2020

Related U.S. Application Data
(60) Provisional application No. 62/832,821, filed on Apr. 11, 2019.
(60) Provisional application No. 62/944,313, filed on Dec. 5, 2019.

(51) Int. Cl. A61F 9/008; A61F 9/007

(52) U.S. Cl. CPC A61F 9/00834; A61F 9/00736; A61F 2009/0087; A61F 2009/00887; A61F 2009/0088; A61F 2009/00842

(57) ABSTRACT

An intraocular lens (IOL) implanted in a patient's eye in a cataract procedure is modified by altering the spatial refractive index profile of the IOL to remove higher order aberrations of the patient's visual system. The higher order aberrations are measured by an aberrometer, and the measured distortions on the cornea are propagated from the corneal surfaces to the IOL plane, and corrected in the IOL. This allows the choice to have high order aberration correction to be an independent choice for the patient, independent of the decision to have cataract surgery. In addition, patients with existing standard IOLs implanted may obtain the benefit of high order aberration correction at any time after implantation.

### CROSS-REFERENCES TO RELATED APPLICATIONS

This application claims priority to and incorporates by reference the entire contents of U.S. Provisional Application No. 62/832821 (Attorney Docket No. JSV7058USPSP1), filed Apr. 11, 2019, and U.S. Provisional Application No. 62/944313 (Attorney Docket No. JSV7058USPSP2), filed Dec. 5, 2019.

### BACKGROUND OF THE INVENTION

### Field of the Invention

This invention relates to post-surgical modification of intraocular lens (IOL), and in particular, it relates to refractive index modification of IOL to achieve higher order correction.

### Description of Related Art

Cataract surgery to remove a cataractous lens and replace it with an artificial intraocular lens (IOL) is a common medical procedure. The standard IOL is a lens with spherical surfaces of the appropriate power to focus an image of distant objects on the retina. The spherical surfaces of such a standard IOL may not provide the ideal image quality on the retina due to aberrations arising from the cornea of the human eye. These aberrations may include asymmetric corneal astigmatism and other higher order aberrations. These are not corrected by a standard IOL.

Currently higher order aberrations are corrected on patients by performing LASIK or PRK refractive surgeries using an excimer laser. These treatments are performed by altering the shape of the corneal surface. Thus, for a patient with an implanted IOL higher order aberration correction would require an additional surgical procedure on the cornea.

Post-surgical shape correction of the IOL by UV photo cross linking and the resulting shape change has been demonstrated and commercialized, for example, by RxSight, Inc.

### SUMMARY

The present invention is directed to a method of adding higher order compensation, via refractive index modification, to a standard IOL that has already been implanted. This enables improvement of the image on the retina to improve vision in a large number (the majority) of cataract patients.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides a process for correcting a patient's vision, which includes: performing a cataract surgery on the patient's eye to remove a cataract lens and implant an intraocular lens (IOL) in the eye; after implanting the IOL, measuring the patient's vision using an aberrometer to determine higher order aberrations of the eye including the IOL, including determining locations of the eye that have the higher order aberrations; mathematically propagating the higher order aberrations to a plane of correction at the IOL; generating a laser treatment plan that will cause the refractive index change in the IOL that corrects the higher order aberrations; and using an ophthalmic laser system to modify a refractive index of the IOL according to the laser treatment plan.

In another aspect, the present invention provides a process for correcting a vision of a patient's eye, which includes: measuring an aberration of the patient's eye using an aberrometer to determine higher order aberrations of the eye including an intraocular lens (IOL) which has been implanted in the eye, including determining locations of the eye that have the higher order aberrations; mathematically propagating the higher order aberrations to a plane of correction at the IOL; generating a laser treatment plan configured to cause a change in a refractive index of the IOL that corrects the higher order aberrations at the plane of correction; and by an ophthalmic laser system, modifying the refractive index of the IOL according to the laser treatment plan.

In another aspect, the present invention provides an ophthalmic surgical laser system, which includes: a laser light source configured to generate a pulsed laser beam; an optical system including one or more moveable optical elements, configured to focus the laser beam and scan the laser beam in a patient's eye; an aberrometer configured to measure an aberration of the patient's eye; and control electronics coupled to the laser light source, the optical system and the aberrometer, and configured to: control the aberrometer to measure aberration of the eye and to determine higher order aberrations of the eye including an intraocular lens (IOL) which has been implanted in the eye, including determining locations of the eye that have the higher order aberrations; mathematically propagate the higher order aberrations to a plane of correction at the IOL; generate a laser treatment plan configured to cause a change in a refractive index of the IOL that corrects the higher order aberrations at the plane of correction; and control the laser light source and the optical system to scan the laser beam in the eye according to the laser treatment plan, wherein the laser beam modifies the refractive index of the IOL.

It is to be understood that both the foregoing general description and the following detailed description are exemplary and explanatory and are intended to provide further explanation of the invention as claimed.

### BRIEF DESCRIPTION OF THE DRAWINGS

FIG. 1 is a flowchart that schematically illustrates an ophthalmic procedure according to an embodiment of the present invention.

FIG. 2 schematically illustrates an ophthalmic surgical laser system which may be used to implement embodiments of the present invention.

FIG. 3 schematically illustrates a system in which embodiments of the present invention can be implemented.

### DETAILED DESCRIPTION OF PREFERRED EMBODIMENTS

Commonly owned, co-pending U.S. patent application Ser. No. 16/375784, filed Apr. 4, 2019, entitled Methods and Systems for Changing a Refractive Property of an Implantable Intraocular Lens (“the '784 application”), describes a “method of altering a refractive property of a crosslinked acrylic polymer material by irradiating the material with a high energy pulsed laser beam to change its refractive index. The method is used to alter the refractive property, and hence the optical power, of an implantable intraocular lens after implantation in the patient's eye. In some examples, the wavelength of the laser beam is in the far red and near IR range and the light is absorbed by the crosslinked acrylic polymer via two-photon absorption at high laser pulse energy. . . . The method can be used to form a Fresnel lens in the optical zone [of the IOL].” (Abstract.) As described in the '784 application, the IOL may be formed of a crosslinked acrylic polymer, and the refractive index modification is achieved through heating of the material. The laser beam may be in the blue range, or the red and near infrared range, in which case the IOL material absorbs the laser light through two-photon absorption. The content of the '784 application is incorporated herein by reference in its entirety.

Embodiments of the present invention provide a method in which the IOL is specifically modified by altering the spatial refractive index profile of the IOL to remove higher order aberrations measured by an aberrometer. The measured distortions on the cornea are propagated from the corneal surfaces to the IOL plane and corrected in the IOL. This allows the choice to have high order aberration correction to be an independent choice for the patient, independent of the decision to have cataract surgery. In addition, patients with existing standard IOLs implanted may obtain the benefit of high order aberration correction at any time after implantation.

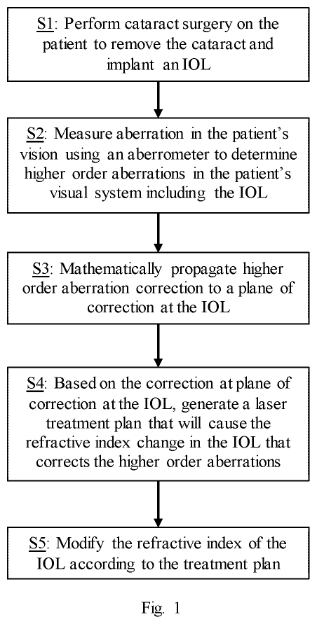

In a process according to an embodiment of the present invention (see FIG. 1), cataract surgery is performed on the patient to remove the cataract and implant an IOL to correct the patient's vision (step S1). This step may be performed using conventional cataract surgery apparatus and techniques. Subsequently, the aberration in the patient's vision is measured using a wavefront aberrometer to objectively determine the higher order aberrations in his/her visual system including the IOL (step S2). In this disclosure, “higher order aberrations” include all aberrations other than piston, tilt, power, and cylinder. The wavefront aberrometer may be, for example, a Hartmann-Shack wavefront sensor, or other types of wavefront sensors known in the art. Techniques of using a wavefront aberrometer to measure higher order aberrations of the visual system are generally known. In this step, the aberrometer determines what surfaces of the visual system are the locations of specific higher order aberrations, such as the cornea anterior and/or posterior surfaces, and/or the IOL itself. Based on this determination, the higher order aberrations are mathematically propagated to the plane of correction at the IOL (step S3). Algorithms and software implementations of propagating an aberration to a given surface in an optical system are generally known in the art. Based on the correction at the plane of correction at the IOL, a laser treatment plan is generated that will cause the refractive index change in the IOL that corrects these higher order aberrations that has been propagated to the plane of correction at the IOL (step S4). An ophthalmic laser system is then used to modify the refractive index of the IOL according to the treatment plan (step S5), for example, using the system and method described in the above-referenced '784 application.

In some embodiments, when the implanted IOL has multifocality, the correction for the higher order aberrations is performed in the IOL while maintaining the existing multifocality.

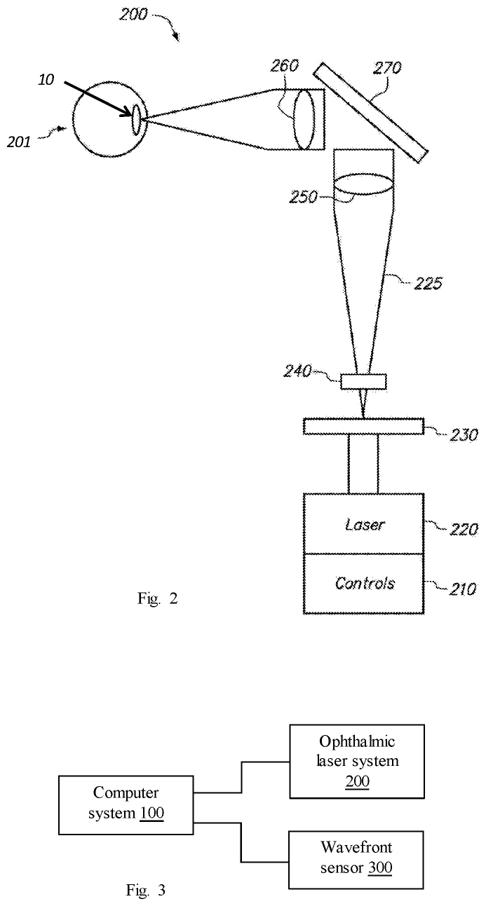

FIG. 2 schematically illustrates an ophthalmic surgical laser system 200 which may be used to implement embodiments of the present invention. The system 200, which can project or scans an optical beam into a patient's eye 201 containing the IOL 10, includes control electronics 210, a laser light source 220, an attenuator 230, a beam expander 240, focusing lenses 250, 260 and reflectors 270. Control electronics 210 may be a computer, microcontroller, etc. with memories storing computer-readable program code to control the operation of various components of the laser system to accomplish the scanning methods described herein. Scanning may be achieved by using one or more moveable optical elements (e.g. lenses 250, 260, reflectors 270) which also may be controlled by control electronics 210, via input and output devices (not shown). Another means of scanning might be enabled by an electro optical deflector device (single axis or dual axis) in the optical path. Although FIG. 2 shows the optical beam directed to a patient's eye, it should be understood that the intraocular lens may be irradiated before placement into the patient's eye in order to customize a refractive property of the intraocular lens.

During operation, the light source 220 generates an optical beam 225 whereby reflectors 270 may be tilted to deviate the optical beam 225 and direct beam 225 towards the patient's eye 201 and particularly into the IOL in order to alter the refractive index of the IOL material. Focusing lenses 250, 260 can be used to focus the optical beam 225 into the patient's eye 201 and the IOL. The positioning and character of optical beam 225 and/or the scan pattern it forms on the eye 201 may be further controlled by use of an input device such as a joystick, or any other appropriate user input device.

Although not shown in FIG. 2, the laser system 200 preferably also includes imaging and visualization sub-systems, such as and without limitation, an optical coherence tomography (OCT) system, a video monitoring system, etc. These sub-systems are used to provide images of and to locate the various anatomical structures of the eye as well as the IOL, which can assist in performance of the various methods described later in this disclosure. Many types of imaging and visualization sub-systems are known in the art and their detailed descriptions are omitted here.

In many embodiments, the light source is a 320 nm to 800 nm pulsed laser source. In many embodiments, the light source 220 is a 320 nm to 800 nm laser source such as an tunable femtosecond laser system or it may be a Nd:YAG laser source operating at the 2nd harmonic wavelength, 532 nm, or 3rd harmonic wavelength, 355 nm.

In operation, the light of the light source is focused and is scanned in the IOL material in order to effect a change of the refractive index in a volume of the material. The shape and volume of the volume whose refractive index is changed is determined by the change in the refractive property of the intraocular lens that is desired.

FIG. 3 schematically illustrates an overall system in which embodiments of the present invention can be implemented. The system includes a computer system 100 which controls the ophthalmic surgical laser system 200 and the wavefront sensor 300. The computer system includes processors and non-volatile memories, where the processors execute computer-readable program code stored in the memories to control the laser system 200 and the wavefront sensor 300 and perform the above-described methods.

It will be apparent to those skilled in the art that various modification and variations can be made in the method and related apparatus of the present invention without departing from the spirit or scope of the invention. Thus, it is intended that the present invention cover modifications and variations that come within the scope of the appended claims and their equivalents.

What is claimed is:

1. A process for correcting a vision of a patient's eye, comprising:
measuring an aberration of the patient's eye using an aberrometer to determine higher order aberrations of the eye including an intraocular lens (IOL) which has been implanted in the eye, including determining locations of the eye that have the higher order aberrations;
mathematically propagating the higher order aberrations to a plane of correction at the IOL;
generating a laser treatment plan configured to cause a change in a refractive index of the IOL that corrects the higher order aberrations at the plane of correction; and
operating an ophthalmic laser system to modify the refractive index of the IOL according to the laser treatment plan.

2. The process of claim 1, further comprising, before the measuring step,
performing a cataract surgery on the patient's eye to remove a cataract lens and implant the IOL in the eye.

3. The process of claim 1, wherein the aberrometer is a wavefront aberrometer.

4. The process of claim 1, wherein the locations of the eye that have the higher order aberrations include a cornea anterior surface, or a cornea posterior surface, or the IOL.

5. The process of claim 1, wherein the higher order aberrations include all aberrations other than piston, tilt, power, and cylinder aberrations.

6. The process of claim 1, wherein the IOL is made of a crosslinked acrylic polymer material.

7. The process of claim 6, wherein the step of modifying the refractive index of the IOL includes heating the crosslinked acrylic polymer material of the IOL with a laser beam generated by the ophthalmic laser system.

8. The process of claim 6, wherein the ophthalmic laser system generates a pulsed laser beam having a wavelength in an far red or near infrared range, and wherein the crosslinked acrylic polymer material of the IOL absorbs the pulsed laser beam via two-photon absorption.

9. An ophthalmic surgical laser system comprising:
a laser light source configured to generate a pulsed laser beam;
an optical system including one or more moveable optical elements, configured to focus the laser beam and scan the laser beam in a patient's eye;
an aberrometer configured to measure an aberration of the patient's eye; and
control electronics coupled to the laser light source, the optical system and the aberrometer, and configured to:
control the aberrometer to measure aberrations of the eye and to determine higher order aberrations of the eye including an intraocular lens (IOL) which has been implanted in the eye, including determining locations of the eye that have the higher order aberrations;
mathematically propagate the higher order aberrations to a plane of correction at the IOL;
generate a laser treatment plan configured to cause a change in a refractive index of the IOL that corrects the higher order aberrations at the plane of correction; and
control the laser light source and the optical system to scan the laser beam in the eye according to the laser treatment plan, wherein the laser beam modifies the refractive index of the IOL.

10. The ophthalmic surgical laser system of claim 9, wherein the aberrometer is a wavefront aberrometer.

11. The ophthalmic surgical laser system of claim 9, wherein the locations of the eye that have the higher order aberrations include a cornea anterior surface, or a cornea posterior surface, or the IOL.

12. The ophthalmic surgical laser system of claim 9, wherein the higher order aberrations include all aberrations other than piston, tilt, power, and cylinder aberrations.

13. The ophthalmic surgical laser system of claim 9, wherein the laser light source is a tunable femtosecond laser configured to generate a pulsed laser beam having a wavelength in an far red or near infrared range.
